(19) United States
(12) Patent Application Publication
(10) Pub. No.: US 2012/0157396 A1
(43) Pub. Date: Jun. 21, 2012

(54) PYRAZINAMIDE FOR IMMUNOSTIMULATION AND THE TREATMENT OF LEISHMANIASES

(75) Inventors: John T. WELCH, Albany, NY (US); Susana MENDEZ, Rockville, MD (US); Michael H. CYNAMON, Dewitt, NY (US)

(73) Assignees: THE RESEARCH FOUNDATION OF STATE UNIVERSITY OF NEW YORK, Albany, NY (US); UNITED STATES GOVERMENT AS REPRESENTED BY THE DEPARTMENT OF VETERANS AFFAIR, Whasington, DC (US); CORNELL UNIVERSITY, Ithaca, NY (US)

(21) Appl. No.: 13/301,128

(22) Filed: Nov. 21, 2011

Related U.S. Application Data
(63) Continuation-in-part of application No. PCT/US2010/035444, filed on May 19, 2010.
(60) Provisional application No. 61/179,471, filed on May 19, 2009.

(51) Int. Cl. A61K 31/4965; A61K 31/7048; A61K 31/7036; A61P 37/04; A61K 31/496; A61K 31/541; A61K 31/555; A61P 33/02; A61K 31/69; A61K 31/661

(52) U.S. Cl. 514/31; 514/255.06; 514/64; 514/39; 514/77; 514/254.07; 514/227.8; 514/184

(57) ABSTRACT

Applicants claim the use of a pyrazine compound of formula I:

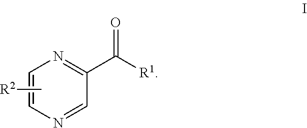

or a salt thereof, for treating or preventing leishmaniases, and diseases and disorders caused by Trypanosoma cruzi or Trypanosoma brucei, and for inducing immunostimulation.

### CROSS REFERENCE TO RELATED APPLICATIONS

This application is a continuation-in-part of International Application Number PCT/US2010/035444, filed May 19, 2010, which published in English as WO 2010/135452 on Nov. 25, 2010, and which claimed priority from U.S. Provisional Application No. 61/179,471, filed May 19, 2009. The entire disclosures of each of the prior applications are hereby incorporated herein by reference.

### FIELD OF THE INVENTION

The invention relates to the use of certain compositions comprising pyrazinamide and pyrazinamide analogs for the treatment and prevention of leishmaniases and diseases and disorders caused by Trypanosoma cruzi and Trypanosoma brucei. The invention also relates to the use of pyrazinamide and pyrazinamide analogs for inducing immunostimulation of leukocytes.

### BACKGROUND OF THE INVENTION

The leishmaniases are a group of insect-transmitted parasitic diseases prevalent worldwide, endemic in 88 countries; 350 million people are at risk and 12 million people are affected. Two million new cases of leishmaniasis are estimated to occur annually, although only 600,000 are officially reported. During the last two decades, it has become increasingly apparent that the leishmaniases are much more prevalent than had been previously suspected. With human migration and vector expansion dramatically affecting the spread of disease, it is found in areas previously considered free of infection. As a result, dramatic outbreaks can occur in locations with previously low levels of infection (i.e. Kabul, more than 200,000 infected). Naïve individuals from the developed world, traveling to endemic areas, are particularly prone to infection. Leishmaniasis has been reported among soldiers deployed to the Middle East during both Gulf wars, as well as to conflicts in Afghanistan and Central America. Humanitarian aid workers traveling in these areas are also at risk.

Leishmaniases are caused by Leishmania, which are protozoan parasites distributed worldwide and transmitted by the bite of an infected sandfly. Multiple species infect humans and cause a spectrum of diseases ranging from self-healing cutaneous ulcers to life-threatening visceral disease. Cutaneous leishmaniasis (CL), while often disfiguring, is generally self-limited. CL can respond to chemotherapy, but resistance has been reported. Visceral leishmaniasis (VL) is a systemic disease marked by fever, weight loss, hepatosplenomegaly, and pancytopenia. The fatality rate of VL approaches 100% without therapy. About 90% of the world's annual new cases are caused by L. donovani in South Asia and Sudan. VL is also the second most common opportunistic infection by tissue protozoa in people with HIV/AIDS. Every year, more than 100,000 cases occur in India alone. Ongoing epidemics of VL kill about 60,000 people each year.

Currently, there are approximately twenty-five licensed compounds with anti-leishmanial effects, but only a few are used in humans. As recently as 2004, liposomal amphotericin B, miltefosine and paromomycin were identified by WHO/TDR as the three most promising drugs in the market. These drugs are not new: amphotericin B has been extensively used for decades as a second line drug for treatment of leishmaniasis (in addition to its antifungal activity), miltefosine was developed long ago as an anticancer agent, and the aminoglycoside paromomycin is over 50 years old. To date, these three agents are, together with antimonials and non-liposomal amphotericin B, the reference chemotherapeutic agents for the leishmaniases.

For six decades, pentavalent antimonials (Sb^{v}) have been used as first-line therapy for leishmaniasis. Sb^{v} are difficult to administer: standard treatment involves 30 days of parenteral therapy in a hospital setting. There is also a growing incidence of drug resistance: in a recent study, only 36% of patients were cured with Sb^{v} alone. Pentamidine, also toxic and difficult to administer, is a second-line drug; unfortunately, resistance is a problem with pentamidine, as well. The only current option for patients with refractory disease is amphotericin B, which, although effective, is also toxic and requires prolonged hospitalization. Lipid formulations of the drug are effective in shorter courses, but are prohibitively expensive. Thus, drawbacks associated with conventional treatment with antimonials and amphotericin B include high host toxicity and differences in susceptibility between strains of the organism. Moreover, the expense of these drugs often precludes their use. Oral miltefosine has recently been approved in India, but it has several safety signals in toxicology studies that may limit its broad usage, such as possible teratogenic side effects, and it is reported as substantially less effective outside of India. With these limitations, the development of safer, inexpensive and widely available treatments continues to be one of the top research priorities for disease control.

Trypanosoma cruzi (T. cruzi) is a species of parasitic trypanosomes. Chagas disease, or American trypanosomiasis, is a serious parasitic ailment in Latin America. The World Bank estimated an annual loss of 2.74 million disability-adjusted life years, representing an economic loss to the countries in which the disease is endemic equivalent to U.S. $6.5 billion per year. Chagas disease is a major parasitic cause of death and hardship, especially in the impoverished regions of the developing world. Chagas disease, widely distributed throughout the Americas, is endemic in 21 countries, from Mexico in the north to Argentina and Chile in the south. According to the World Health Organization, there are 16 to 18 million people already infected and some 100 million (25% of the Latin American population) at risk of becoming infected, with around 60,000 people dying every year.

At present, acute cases of trypanosomal diseases are treated with nifurtimox and benznidazole, but there is currently no effective therapy for chronic cases. Both compounds have low efficacy and severe side effects, particularly in adult patients.

Trypanosoma brucei (T. brucei) is a parasitic protist species that causes African trypanosomiasis (or sleeping sickness) in humans and nagana in animals in Africa. African trypanosomiasis is endemic to sub-Saharan Africa. There is an urgent need for the development of new drug therapies as current treatments can prove fatal to the patient as well as the trypanosomes.

### SUMMARY OF THE INVENTION

One aspect of the invention relates to a method of treating or preventing leishmaniases, or diseases or disorders caused by Trypanosoma cruzi or Trypanosoma brucei, comprising administering to a patient an efficacious amount of a pyrazine compound of formula I:

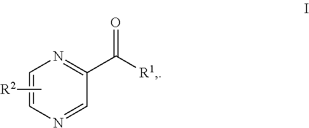

or a salt thereof, wherein R¹ is chosen from NR⁴R⁵ and OR³; R² is chosen from H and halogen; R³ is chosen from H and C₁ to C₂₀ alkyl; and R⁴ and R⁵ are individually chosen from H, NH₂, C₁ to C₂₀ alkyl, oxaalkyl, and heterocyclylalkyl, or taken together R⁴ and R⁵, together with the nitrogen to which they are attached, form a heterocyclic ring.

A second aspect of the invention relates to a method for inducing immunostimulation of leukocytes of a patient in the need of such immunostimulation, comprising administering to the patient an efficacious amount of a pyrazine compound of formula I, or a salt thereof.

### BRIEF DESCRIPTION OF THE FIGURES

FIG. 1. The leishmanicidal effect of PZA. Effect of PZA (100 μM) on the survival of L. major amastigotes within J774 cells at different time points after infection. Data are expressed as percentage survival compared to the untreated control (100% survival). Values are means±standard deviation of at least three independent determinations.

FIG. 2. A. PZA modifies the course of L. major infection. Mean lesion diameter in C57BL/6 mouse ears infected with L. major and treated with PZA versus control (n=6-12 ears±SEM). *: Statistically significant, P=0.0001 when compared with the PZA-treated groups. Numbers represent treatment in days post infection. B. Ear parasite burden per ear (n=4-6±SEM) for C57BL/6 mouse ears infected with L. major and treated with PZA (A: 900 mg/kg, B: 450 mg/kg, C: 150 mg/kg, D: control). Parasite burden was estimated by limiting dilution at 6 weeks post infection (open bars) and 12 weeks post infection (gray bars). *: Statistically significant, P=0.008 when compared with the PZA-treated groups. C. Body weights of experimental mice (n=4) seven weeks post infection.

FIG. 3. PZA treatment of J774 cells increased expression of surface markers. Mean Fluorescence Intensity (MFI) for CD80 (A), CD86 (B) and MHC Class II (C) determined by flow cytometry in J774 cells infected with L. major and treated with 10 and 100 μM PZA. A group of uninfected cells were treated with 100 ng/ml LPS and 10 IU IFN-γ as a positive control of activation. The level of marker expression was also determined in untreated, L. major-infected cells. Unstimulated cell expression fluorescence is also shown. Data are expressed as average MFI±standard deviation from n=3 independent experiments. *: Statistically significant P≦0.001, **: Statistically significant P≦0.0001 when compared with untreated, L. major-infected control group.

FIG. 4. PZA decreases parasite burden in VL. Parasite burden in livers of mice infected with L. donovani and treated with PZA as described at three weeks post infection. *P=0.001.

FIG. 5. PZA increases proliferation in treated splenocytes. Positive proliferation (measured as loss of CFSE staining at day 5) at 3 wk post infection in CFSE-stained splenocytes from C75BL/6 mice infected with L. donovani and treated with drug vehicle (3.8% DMSO, control) or increasing concentrations of PZA (n=3±SD), following in vitro restimulation with 25 μg/ml leishmanial antigen or 2 μg/ml concanavalin A.

FIG. 6. Effect of PZA analogs on J774 cells infected with L. major at 48 hr. Data expressed as % survival with respect to untreated control (100% survival). Values are means±SD of at least 3 experiments.

FIG. 7. 5-Cl PZA prevents disease. Lesion size in mice ears infected with L. major and treated with DMSO vehicle (control) or PZA. Numbers represent treatment on days post infection.

FIG. 8. Effect of PZA on J774 cells infected with Leishmania 48 hr post treatment. Data expressed as % survival compared to untreated control (100% survival). Mean±SD of 3 independent determinations. PZA killed old-world and new-world VL leishmanial species (reduced liver parasites 2-4 log 10, prevented hepatomegaly and splenomegaly in mouse models), as well as species that cause cutaneous disease.

FIG. 9. A. Effect of 5-Chloro PZA on J774 cells infected with Leishmania, 48 hr post treatment. Data expressed as % survival compared to untreated control (100% survival). Mean±SD of 3 independent determinations. 5-Chloro PZA was 10-fold more active than PZA itself at killing a variety of old- and new-world leishmanial species, with MIC ranging between 1-13 μM. B. Cytokine IL-2 production—5-Chloro PZA stimulated 2× more proinflammatory cytokines than PZA, even in Leishmania-infected macrophages (similar results with IFNγ, TNFα, NO).

FIG. 10. PZA increases proinflammatory cytokine production in J774 cells. IL-10, IL-12, TNF-α, and nitric oxide production determined by ELISA (cytokines) and Griess test (nitric oxide) in J774 cells infected or not with L. major and treated with 0.1 and 1 μg/ml amphotericin B. Uninfected cells were treated with 10 U IFN-γ and 100 ng/ml LPS as a positive control of activation. Unstimulated cell cytokine levels are shown. Data are expressed as mean±standard deviation of n=3 determinations. *: Statistically significant, P=0.0001 if compared with the group infected with L. major that did not receive the drug.

FIG. 11. A. The immunostimulatory effect of PZA is independent of TLR engagement. Mean fluorescence values for the expression of CD80, CD86, MHC Class I and MHC Class II and B. IL-12, IL-10 and iNOS expression determined by flow cytometry in bone marrow-derived dendritic cells from TLR-2/-4 double knock out mice, infected or not with L. major, and treated with 10 and 100 μM PZA. Uninfected cells were treated with 10 U IFN-γ/100 ng/ml LPS as a positive control of activation. Unstimulated, untreated cells are also shown. Data are expressed as mean±standard deviation of n=3 determinations. *: Statistically significant P≦0.03; **: Statistically significant P≦0.005; ***: Statistically significant, P≦0.0001 when compared to the group infected with L. major and untreated.

FIG. 12. A. Lesion due to L. major in a human arm. B. Lesion in a C57B1/6 mouse ear. C. Typical course of L. major infection in the C57B1/6 mice. Data expressed as mean±standard deviation of lesion size (mm).

FIG. 13. Mean fluorescence values for the expression of CD80, CD86, MHC Class I and MHC Class II determined by flow cytometry in J774 cells infected with L. major and treated with 10 and 100 μM PZA (A) and 5-Cl PZA (B). Uninfected cells were treated with 10 U IFN-γ and 100 ng/ml LPS as a positive control of activation. Marker expression was also determined in L. major infected untreated macrophages and in untreated cells.

FIG. 14. IL-12, TNF-α, IL-10 and NO production determined by ELISA (cytokines) and Griess test (NO) in J774 cells infected or not with L. major and treated with 10 and 100 μM PZA (A) or 5-Cl PZA (B). Uninfected cells were treated with 10 U IFN-γ and 100 ng/ml LPS as a positive control of activation. Unstimulated cell cytokine levels are also shown.

FIG. 15. IL-10, IL-12, TNF-α□, and NO production determined by ELISA (cytokines) and Griess test (NO) in J774 cells infected or not with L. major and treated with 0.1 and 0.5 μg/m amphotericin B. Uninfected cells were treated with 10 U IFN-γ and 100 ng/ml LPS as a positive control of activation. Unstimulated cell cytokine levels are shown.

### DETAILED DESCRIPTION OF THE INVENTION

In one aspect, the invention relates to a method of treating or preventing leishmaniases, or a disease or disorder caused by Trypanosoma cruzi or Trypanosoma brucei, comprising administering to a patient an efficacious amount of a pyrazine compound of formula I:

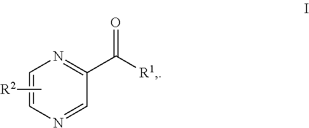

or a salt thereof, wherein R¹ is chosen from NR⁴R⁵ and OR³; R² is chosen from H and halogen; R³ is chosen from H and C₁ to C₂₀ alkyl; and R⁴ and R⁵ are individually chosen from H, NH₂, C₁ to C₂₀ alkyl, oxaalkyl, and heterocyclylalkyl, or taken together R⁴ and R⁵, together with the nitrogen to which they are attached, form a heterocyclic ring. In some embodiments, R¹ is chosen from a 5- or 6-membered heterocycle (such as a nitrogen-attached pyrrolidine). In some embodiments R² is chosen from fluorine, chlorine, and bromine. In some embodiments, R³ is chosen from C₁ to C₁₂ alkyl, or from C₁ to C₆ alkyl, or from C₁ to C₃ alkyl. In some embodiments, R⁴ and/or R⁵ are a morpholinoalkyl group, or C₁ to C₁₂ alkyl, C₁ to C₆ alkyl, or C₁ to C₃ alkyl, and in said alkyl residues, one or more carbon atoms (and their associated hydrogens) may optionally be replaced by oxygen.

The following are some examples of embodiments that are encompassed by formula I:

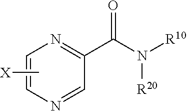

| No. | X | R¹⁰ | R²⁰ |
|---|---|---|---|
| 1 | Cl | (CH₂)₄ | |
| 2 | Cl | Et | Et |
| 3 | Cl | C₆H₁₁ | C₆H₁₁ |
| 4 | Cl | C₂H₄OCH₃ | H |
| 5 | Cl | C₅H₉ | H |
| 6 | Cl | C(CH₃)₃ | H |
| 7 | H | CH₂N(CH₂)₄ | H |
| 8 | H | CH₂N[(CH₂)₄O] | H |

In another aspect, the invention relates to a method of treating leishmaniases, comprising administering to a patient an efficacious amount of a pyrazine compound of formula I or a salt thereof.

In another aspect, the invention relates to a method of treating leishmaniases, comprising administering to a patient an efficacious amount of a combination of a compound of formula I or a salt thereof and one or more compounds or salts thereof selected from meglumine antimoniate, sodium stibogluconate, amphotericin B, paromomycin, pentamidine, miltefosine, and ketoconazole.

In any embodiments of the invention that include amphotericin B, said compound may be used in the form of uncomplexed amphotericin B, liposomal formulations of amphotericin B, such as AmBisome™ or Fungisome™, amphotericin B complexed with cholesteryl sulfate, such as Amphotec™, or amphotericin B complexed with phospholipids, such as Abelcet™.

In another aspect, the invention relates to methods of treating or preventing diseases or disorders caused by Trypanosoma cruzi comprising administering to a patient an efficacious amount of a pyrazine compound of formula I or a salt thereof.

In another aspect, the invention relates to methods of treating or preventing diseases or disorders caused by Trypanosoma cruzi, comprising administering to a patient an efficacious amount of a combination of a compound of formula I or a salt thereof and one or more compounds or salts thereof selected from nifurtimox and benznidazole.

Another aspect of the invention relates to methods of treating or preventing diseases or disorders caused by Trypanosoma brucei, comprising administering to a patient an efficacious amount of a pyrazine compound of formula I or a salt thereof.

In another aspect, the invention relates to methods of treating or preventing diseases or disorders caused by Trypanosoma brucei, comprising administering to a patient an efficacious amount of a combination of a compound of formula I or a salt thereof and one or more compounds or salts thereof selected from Nifurtimox, pentamidine, eflornithine, and melarsoprol.

In one aspect, the invention relates to a method of treating or preventing leishmaniases, or a disease or disorder caused by Trypanosoma cruzi or Trypanosoma brucei, comprising administering to a patient an efficacious amount of pyrazinamide or a salt thereof.

In another aspect, the invention relates to a method of treating or preventing leishmaniases, or a disease or disorder caused by Trypanosoma cruzi or Trypanosoma brucei, comprising administering to a patient an efficacious amount of 5-chloro pyrazinamide or a salt thereof.

In another aspect, the invention relates to methods of treating or preventing leishmaniases, or a disease or disorder caused by Trypanosoma cruzi or Trypanosoma brucei, comprising administering to a patient an efficacious amount of a compound of formula I, wherein at least one of R⁴ and R⁵ is morpholinomethyl.

In one aspect, the invention relates to a method of treating or preventing leishmaniases, or a disease or disorder caused by Trypanosoma cruzi or Trypanosoma brucei, comprising administering to a patient an efficacious amount of a compound of formula IA, IB, or IC.

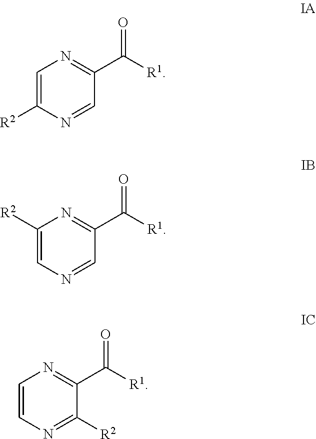

In another aspect, the invention relates to a method for inducing immunostimulation of leukocytes of a patient in the need of such immunostimulation comprising administering to the patient an efficacious amount of a pyrazine compound of formula I:

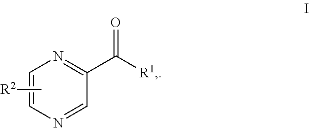

or a salt thereof, wherein R¹ is chosen from NR⁴R⁵ and OR³; R² is chosen from H and halogen; R³ is chosen from H and C₁ to C₂₀ alkyl; and R⁴ and R⁵ are individually chosen from H, NH₂, C₁ to C₂₀ alkyl, oxaalkyl, and heterocyclylalkyl, or taken together R⁴ and R⁵, together with the nitrogen to which they are attached, form a heterocyclic ring. In some embodiments, R¹ is chosen from a 5- or 6-membered heterocycle (such as a nitrogen-attached pyrrolidine). In some embodiments R² is chosen from fluorine, chlorine, and bromine. In some embodiments, R³ is chosen from C₁ to C₁₂ alkyl, or from C₁ to C₆ alkyl, or from C₁ to C₃ alkyl. In some embodiments, R⁴ and/or R⁵ are a morpholinoalkyl group, or C₁ to C₁₂ alkyl, C₁ to C₆ alkyl, or C₁ to C₃ alkyl, and in said alkyl residues, one or more carbon atoms (and their associated hydrogens) may optionally be replaced by oxygen.

In another aspect, the invention relates to a method for inducing immunostimulation of leukocytes of a patient in the need of such immunostimulation comprising administering to the patient an efficacious amount of pyrazinamide or a salt thereof.

In another aspect, the invention relates to a method for inducing immunostimulation of leukocytes of a patient in the need of such immunostimulation comprising administering to the patient an efficacious amount of 5-chloro pyrazinamide or a salt thereof.

In another aspect, the invention relates to a method for inducing immunostimulation of leukocytes of a patient in the need of such immunostimulation comprising administering to the patient an efficacious amount of a compound of formula I, wherein at least one of R⁴ and R⁵ is morpholinomethyl.

In another aspect, the invention relates to a method for inducing immunostimulation of leukocytes of a patient in the need of such immunostimulation comprising administering to the patient an efficacious amount a compound of formula IA, IB, or IC.

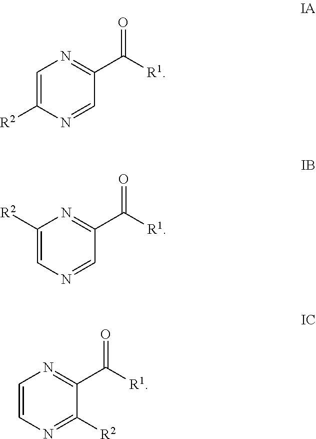

The terms “method[s] of treating or preventing” mean amelioration, prevention or relief from the symptoms and/or effects associated with lipid disorders. The term “preventing” as used herein refers to administering a medicament beforehand to forestall or obtund an acute episode. The person of ordinary skill in the medical art (to which the present method claims are directed) recognizes that the term “prevent” is not an absolute term. In the medical art it is understood to refer to the prophylactic administration of a drug to substantially diminish the likelihood or seriousness of a condition, and this is the sense intended in applicants' claims. As used herein, reference to “treatment” of a patient is intended to include prophylaxis.

The term “halogen” means fluorine, chlorine, bromine or iodine. In one embodiment, halogen may be fluorine or chlorine.

Alkyl is intended to include linear or branched hydrocarbon structures and combinations thereof. A combination would be, for example, cyclopropylmethyl. Lower alkyl refers to alkyl groups of from 1 to 6 carbon atoms. Examples of lower alkyl groups include methyl, ethyl, propyl, isopropyl, butyl, s- and t-butyl and the like. Preferred alkyl groups are those of C₂₀ or below. Cycloalkyl is a subset of alkyl and includes cyclic hydrocarbon groups of from 3 to 8 carbon atoms. Examples of cycloalkyl groups include c-propyl, c-butyl, c-pentyl, norbornyl and the like.

Heterocycle means a cycloalkyl or aryl carbocycle residue in which from one to three carbons is replaced by a heteroatom selected from the group consisting of N, O and S. The nitrogen and sulfur heteroatoms may optionally be oxidized, and the nitrogen heteroatom may optionally be quaternized. Unless otherwise specified, a heterocycle may be non-aromatic or aromatic. Examples of heterocycles that fall within the scope of the invention include pyrrolidine, pyrazo le, pyrrole, indole, quinoline, isoquinoline, tetrahydroisoquinoline, benzofuran, benzodioxan, benzodioxole (commonly referred to as methylenedioxyphenyl, when occurring as a substituent), tetrazole, morpholine, thiazole, pyridine, pyridazine, pyrimidine, thiophene, furan, oxazole, oxazoline, isoxazole, dioxane, tetrahydrofuran and the like. It is to be noted that heteroaryl is a subset of heterocycle in which the heterocycle is aromatic. Examples of heterocyclyl residues additionally include piperazinyl, 2-oxopiperazinyl, 2-oxopiperidinyl, 2-oxo-pyrrolidinyl, 2-oxoazepinyl, azepinyl, 4-piperidinyl, pyrazolidinyl, imidazolyl, imidazolinyl, imidazolidinyl, pyrazinyl, oxazolidinyl, isoxazolidinyl, thiazolidinyl, isothiazolyl, quinuclidinyl, isothiazolidinyl, benzimidazolyl, thiadiazolyl, benzopyranyl, benzothiazolyl, tetrahydrofuryl, tetrahydropyranyl, thienyl, benzothienyl, thiamorpholinyl, thiamorpholinylsulfoxide, thiamorpholinylsulfone, oxadiazolyl, triazolyl and tetrahydroquinolinyl.

Oxaalkyl refers to alkyl residues in which one or more carbons (and their associated hydrogens) have been replaced by oxygen. Examples include methoxypropoxy, 3,6,9-trioxadecyl and the like. The term oxaalkyl is intended as it is understood in the art [see Naming and Indexing of Chemical Substances for Chemical Abstracts, published by the American Chemical Society, 196, but without the restriction of 127(a)], i.e. it refers to compounds in which the oxygen is bonded via a single bond to its adjacent atoms (forming ether bonds); it does not refer to doubly bonded oxygen, as would be found in carbonyl groups. Similarly, thiaalkyl and azaalkyl refer to alkyl residues in which one or more carbons has been replaced by sulfur or nitrogen, respectively. Examples include ethylaminoethyl and methylthiopropyl.

It is understood that any alkyl, alkenyl, alkynyl, cycloalkyl and cycloalkenyl moiety described herein can also be an aliphatic group, an alicyclic group or a heterocyclic group. An “aliphatic group” is non-aromatic moiety that may contain any combination of carbon atoms, hydrogen atoms, halogen atoms, oxygen, nitrogen or other atoms, and optionally contain one or more units of unsaturation, e.g., double and/or triple bonds. An aliphatic group may be straight chained, branched or cyclic and preferably contains between about 1 and about 24 carbon atoms, more typically between about 1 and about 12 carbon atoms. In addition to aliphatic hydrocarbon groups, aliphatic groups include, for example, polyalkoxyalkyls, such as polyalkylene glycols, polyamines, and polyimines, for example. Such aliphatic groups may be further substituted. It is understood that aliphatic groups may be used in place of the alkyl, alkenyl, alkynyl, alkylene, alkenylene, and alkynylene groups described herein.

Substituents Rⁿ are generally defined when introduced and retain that definition throughout the specification and in all independent claims.

As used herein, and as would be understood by the person of skill in the art, the recitation of “compound(s)”—unless expressly further limited—is intended to include salts, solvates and inclusion complexes of the compound(s) mentioned. Thus, for example, the recitation of “compounds” as depicted above includes the listed compounds. In a particular embodiment, the term “compound(s)” refers to the compound or a pharmaceutically acceptable salt thereof The term “pharmaceutically acceptable salt” refers to salts prepared from pharmaceutically acceptable non-toxic acids or bases including inorganic acids and bases and organic acids and bases. Pyrazinamide is not usually found in a salt form, but a salt can be made with a strong acid such as HCl. Unless otherwise stated or depicted, structures depicted herein are also meant to include all stereoisomeric (e.g., enantiomeric, diastereomeric, and cis-trans isomeric) forms of the structure; for example, the R and S configurations for each asymmetric center, (Z) and (E) double bond isomers, and (Z) and (E) conformational isomers. Therefore, single stereochemical isomers as well as enantiomeric, diastereomeric, and cis-trans isomeric (or conformational) mixtures of the present compounds are within the scope of the invention. Unless otherwise stated, all tautomeric forms of the compounds of the invention are within the scope of the invention. Additionally, unless otherwise stated, structures depicted herein are also meant to include compounds that differ only in the presence of one or more isotopically enriched atoms. For example, compounds having the present structures except for the replacement of hydrogen by deuterium or tritium, or the replacement of a carbon by a ¹³C- or ¹⁴C-enriched carbon are within the scope of this invention. Such compounds are useful, for example, as analytical tools or probes in biological assays.

All of the compounds falling within the foregoing parent genus and its subgenera are useful against leishmaniases, but not all of the compounds are novel. In particular, certain known species fall within the genus I, although no anti-leishmania utility has been suggested for these species. It may be found upon examination that compounds that have been excluded from the claims are patentable to the inventors in this application; it may also be found that additional species and genera not presently excluded are not patentable to the inventors in this application. In either case, the exclusion of species and genera in applicants' claims are to be considered artifacts of patent prosecution and not reflective of the inventors' concept or description of their invention. The invention is all methods for treating or preventing leishmaniases, or a disease or disorder caused by Trypanosoma cruzi or Trypanosoma brucei, and all methods of inducing immunostimulation of leukocytes of a patient in the need of such immunostimulation, comprising administering a compound of formula I, except any of those methods that may be in the public's possession.

During development of the instant invention, it was found that PZA has anti-leishmanial effects in vitro on both promastigotes and amastigotes, the latter being less sensitive to the drug. Most interestingly, PZA dramatically decreased lesion development and parasite burden in C57BL/6 mice infected with Lm. Finally, it is shown that PZA increases activation of infected macrophages as assessed by increased expression of co-stimulatory molecules and secretion of IL-12. These results not only indicate that PZA constitutes a very promising alternative therapy to leishmaniasis, but also suggest that the compound induces never before reported collateral immunostimulation.

Leishmania spp., the causative organism in leishmaniasis, is one of three distinct kinetoplastids that cause human disease. The other two kinetoplastids are Trypanosoma cruzi (Chagas disease), and Trypanosoma brucei (African trypanosomiasis, or African sleeping sickness). In view of the proven effects of PZA on Leishmania major, it is also expected that PZA will be effective in the treatment and prevention of diseases and disorders caused by T. cruzi and T. brucei.

### PZA has Anti-Leishmanial Effect In Vitro

The effect of PZA was first investigated with promastigotes of L. major. PZA was diluted in medium and added to cultures. After 48 h, parasite multiplication was determined by counting parasites in culture. L. major promastigotes treated with PZA exhibited a reduction in cell proliferation. The Microbial Inhibitory Concentration 50 (MIC₅₀) was established as 16.2 μg/ml (16.1 μM) (Table I). Intracellular amastigotes appeared to be less sensitive to the effect of both amphotericin B and PZA than the extracellular forms (MIC₅₀=10.2 μg/ml or 8.2 μM). Incubation of L. major with 1 μg/ml amphotericin B (as a positive control) caused 100% mortality of promastigotes and 90% of amastigotes.

TABLE I
Effect of PZA on L. major and J774 cell survival. Amphotericin B (1 μg/ml) was included in the experiment as a
control. After 48 h, parasite and cell survival were determined. Data are expressed as percentage of survival compared
to the untreated control (100% survival). Values are means ± standard deviation of at least three independent determinations.

| | PZA (μg/ml) | | | | | | | |
|---|---|---|---|---|---|---|---|---|
| | 1 | 12.5 | 50 | 100 | 200 | 1000 | MIC₅₀ (μg/ml and μM) | Amphotericin B |
| Promastigote | 88 ± 18 | 50 ± 18 | 38 ± 14 | — | 20 ± 10 | 10 ± 12 | 16.2 and 16.1 | 0 |
| Amastigote | 89 ± 12 | 47 ± 12 | — | 23 ± 10 | — | 20 ± 15 | 10.2 and 8.2 | 10 ± 5 |
| Uninfected J774 cells | 100 ± 11 | 100 ± 10 | 100 ± 12 | 100 ± 15 | 85 ± 12 | 0 | 524.8 and 425.6 | 95 ± 16 |

—: Not determined

The potential cytotoxicity of PZA toward the mammalian cell line was determined by co-incubation of PZA with uninfected J774 cells for 48 h (104 cells/well) in 96-well culture plates. It was found that PZA was cytotoxic at the highest concentration tested (1 mg/ml), resulting in 100% mortality of the macrophages. At 200 μg/ml, the compound started to show signs of toxicity, resulting in mortality of 25% of the cell monolayer. The MIC₅₀ was established as 425.6 μg/ml. The control drug amphotericin B caused 5% mortality of the cell monolayer at the concentration tested (1 μg/ml).

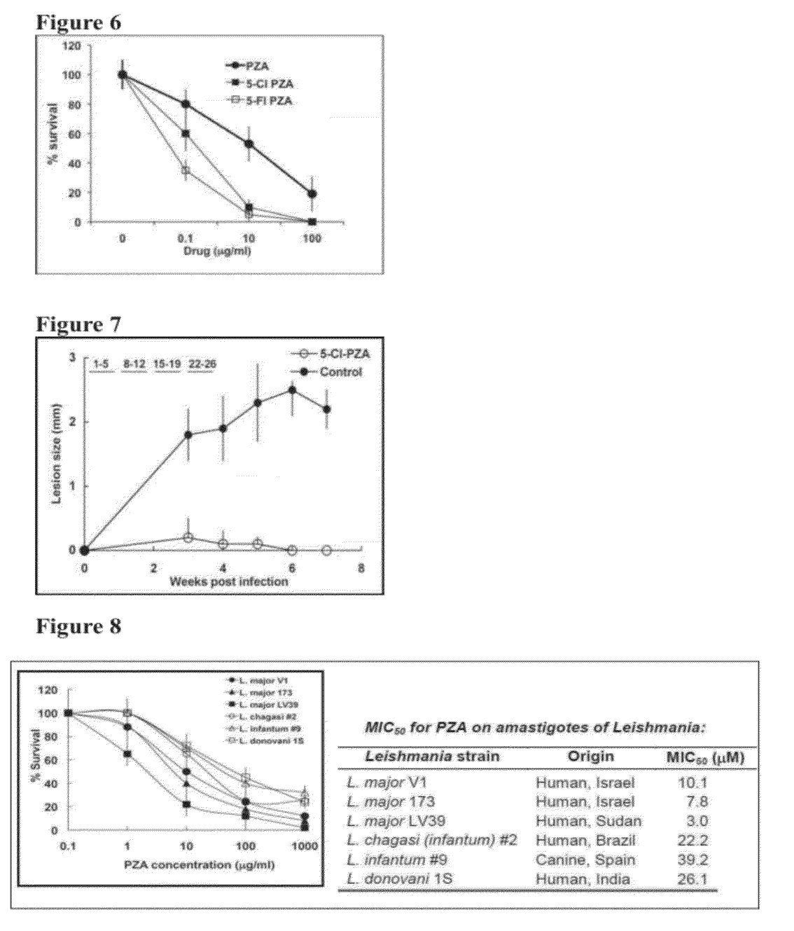

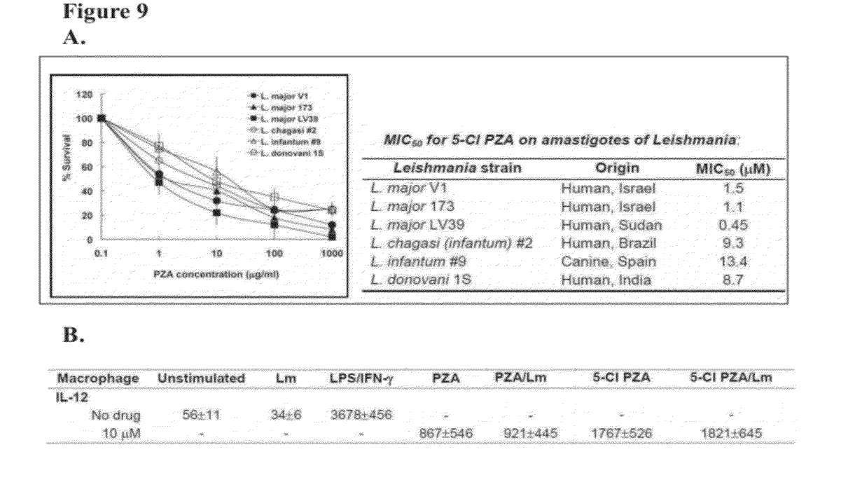

Anti-leishmanial activity of 5-chloro (Cl) and 5-fluoro (Fl) PZA on L. major was also investigated. As with PZA, amastigotes showed a dose-dependent decrease in growth within J774 cells if treated with analogs (FIG. 6). MIC₅₀ for the analogs, however, was reduced by 5 and 100 fold respectively (Table Ia), suggesting that, as in TB, PZA analogs are more efficient drugs than PZA. Finally, we assessed efficacy of 5-Cl PZA in a model of cutaneous disease. C57BL/6 mice were infected in the ears with 5×10⁵ L. major, and treated with 150 mg/ml. As with PZA, oral administration of 5-Cl PZA produced a significant reduction in average lesion size in treated mice compared to untreated controls (FIG. 7) was even more effective than PZA.

TABLE Ia
MIC₅₀ for PZA analogs.

| Drug | MIC₅₀ (μM) |
|---|---|
| PZA | 9.5 |
| 5-Cl PZA | 2.1 |
| 5-Fl PZA | 0.08 |

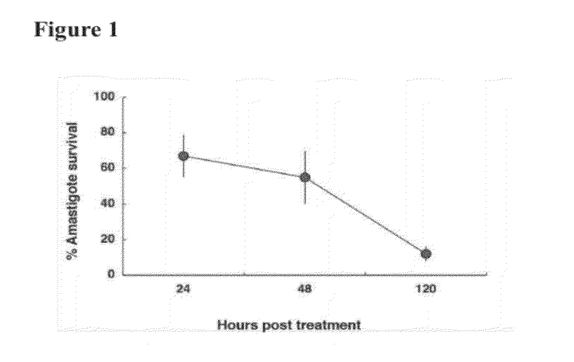

To distinguish between leishmaniostatic and leishmanicidal effects, a time course survival curve was generated for J774 cultures infected with L. major. FIG. 1 shows parasite loads in macrophages at 24, 48 and 120 h post treatment with 100 μM PZA. Interestingly, about 90% of L. major parasites were efficiently eliminated from the cells after 120 hours, indicating that PZA is a leishmanicidal drug.

### PZA Significantly Reduces Clinical Disease and Parasite Burden in Infected Mice

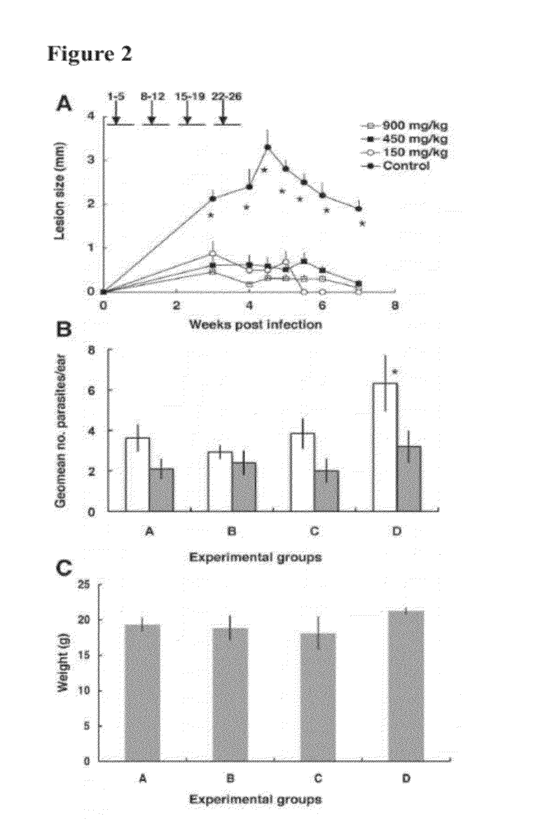

Mouse Model of CL. To assess the in vivo efficacy of PZA in a relevant in vivo model of cutaneous leishmaniasis, C57BL/6 mice were intradermally infected with 5×10⁵ L. major parasites per ear (n=6 mice, 12 ears). One day after infection, mice were treated orally with PZA at several concentrations (900-150 mg/kg) for four weeks, five days a week. As shown in FIG. 2A, the oral administration of PZA produced a significant (P=0.0001) reduction of the average lesion size on all treated groups compared with untreated mice at all time points. Parasite burden in ears was determined at 5 weeks post infection in all experimental groups. FIG. 2B shows that PZA treatment dramatically decreased parasite burden in infected ears at week 6 (100-fold, P=0.008) if compared to the untreated control. Parasite burdens were also comparable among all experimental groups when determined after healing, at 12 weeks post infection. Finally, PZA treatment did not affect the growth of the experimental animals since no significant differences in body weight were found at week seven post infection (FIG. 2C).

Mouse Models of VL. Despite relative resistance to visceral disease, systemic infection of mice with L. donovani provided a complementary model of VL pathogenesis and therapy, and is commonly used as a first investigational stage for drug testing.

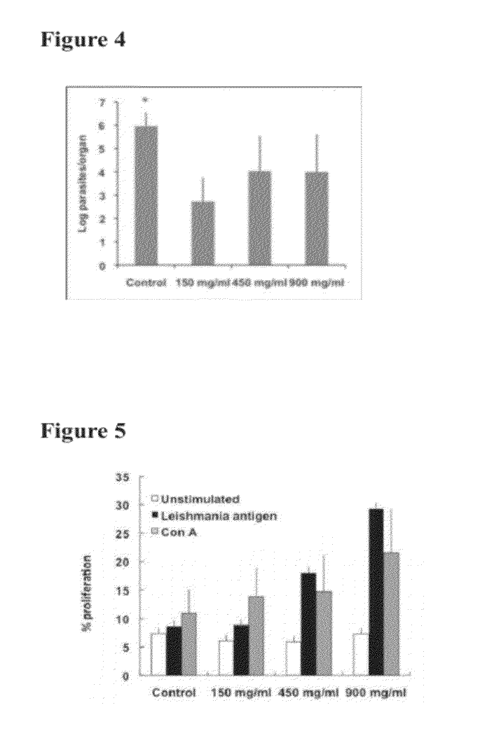

To induce visceral leishmaniasis, we inoculated mice (n=8) intravenously with 5×10⁶ L. donovani 1S and treated them with PZA (900-150 mg/kg) for 3 wks, 5 days/wk. The lowest dose was equivalent to dose of PZA used in humans (2 g/day). A group of mice treated with vehicle (3.8% DMSO in water) was included as a control. C57BL/6 mice infected with L. donovani typically show detectable parasites in liver and spleens for 8-12 wk. Infected mice do not demonstrate typical pathology seen in human VL, but may lose weight and develop hepatomegally and splenomegaly during the course of the infection. We euthanized mice 3 wks after the initiation of the experiment and determined total body weight, liver and spleen weights, and number of parasites in livers. At this early time point, no significant differences were found in total body weight. However, the size of livers and spleens was significantly reduced (P≦0.05) in mice treated with PZA (not shown). Moreover, liver parasites were significantly decreased (P=0.04) in mice treated with PZA at all doses tested (FIG. 4).

Analysis of lymphoproliferative responses of splenocytes from L. donovani-infected mice indicates that PZA treatment enhances the host immune responses against Leishmania. VL typically induces T cell anergy toward leishmanial antigens. We however found that splenocytes from L. donovani-infected mice treated with PZA recovered their ability to proliferate to leishmanial antigens (25 mg/ml) and concanavalin A (2 mg/ml) in a dose depending manner, indicating that PZA treatment not only decreased parasite burden, but also improved the ability of the host cells to respond to the parasite infection (FIG. 5).

PZA Increases J774 Cell Activation as well as Release of Proinflammatory Cytokines and Nitric Oxide

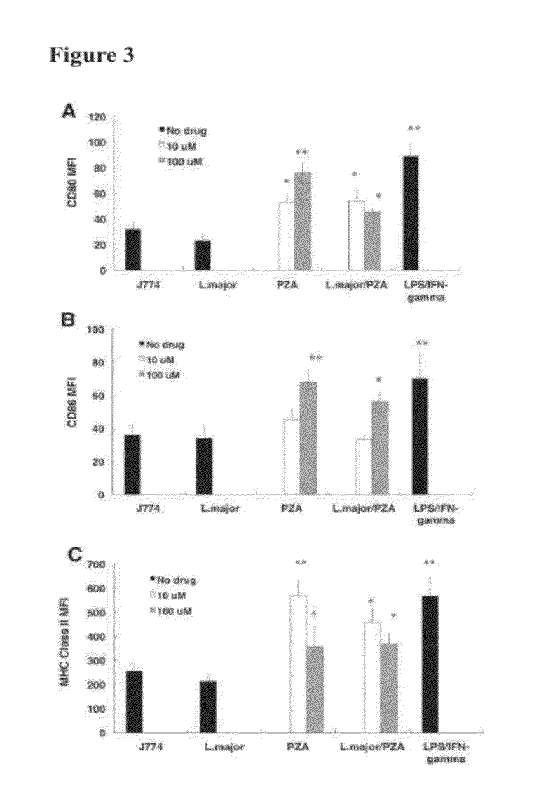

Because of the strong in vivo effect of PZA, we studied the effect of treatment on macrophages. First, we looked at activation markers and cytokine secretion by J774 cells (a murine macrophage cell line) following treatment with PZA (10 and 100 μM) in the presence or absence of L. major infection (1:5 macrophage:parasite ratio) were examined. As a positive control, cells were exposed to a mixture of IFN-γ and LPS, known to increase the level of activation. Expression of surface molecules was determined in unstimulated cells, and on infected, untreated cultures. Twenty-four hours after infection and/or treatment, cells were collected and stained with fluorescent antibodies against the co-stimulatory molecules CD80 and CD86, as well as MHC Class II, and analyzed by flow cytometry. FIG. 3 shows that drug treatment increased expression of the costimulatory molecules C80, C86 as well as MHC Class II, suggesting that treatment alone increases the ability of the macrophage to present antigen. L. major-infected J774 downregulated the expression of costimulatory molecules as well as Class II MHC molecules if compared to infected, untreated controls, a phenomenon typically associated with L. major infection. Interestingly, treatment of L. major-infected cells with PZA rescued the ability of the cell line to upregulate all surface markers studied at the same level than cells treated with the drug alone.

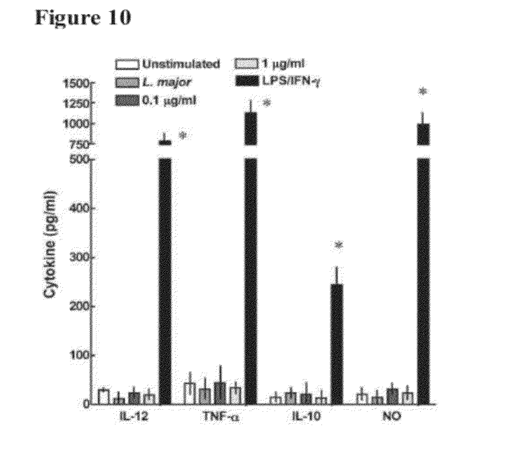

The ability of J774 cells to produce cytokines in response to infection and/or treatment was also determined. Cells were infected, treated or activated as described above. The amount of the proinflammatory cytokines IL-12 and TNF-α (implicated in Thl response and parasite killing), as well as the repressive cytokine IL-10, was determined by ELISA in the culture supernatants. The level of nitric oxide was also determined in the same supernatants. PZA treatment alone increased cytokine production, especially IL-12 and TNF-α and NO release. Cytokine production was also increased in the wells treated with PZA and infected with L. major compared with L. major alone, suggesting again that the immune response is enhanced by PZA in infected cells (Table II). Treatment also slightly increased IL-10 production, although the response of the cell line to the drug was dominated by the release of proinflammatory factors. Finally, to test the specificity of the immune activation by PZA, we determined the effect that amphotericin B treatment had on activation and cytokine expression by J774 cells. Treatment of J774 cells with the drug did not result in activation at the doses tested (FIG. 10).

TABLE II
IL-12, TNF-α, IL-10 and nitric oxide production determined by ELISA (cytokines) and Griess test
(nitric oxide) in J774 cells infected with L. major and treated with 10 and 100 μM PZA. A group
of uninfected cells were treated with 100 ng/ml LPS and 10 IU IFN-γ as a positive control of
activation. Cytokine levels (pg/ml ± standard deviation) were also determined in L. major
infected macrophages, and in untreated cells.

| Drug concentration (μM) | Unstitaulated | L. major | PZA | L. major/PZA | LPS/IFN-γ |
|---|---|---|---|---|---|
| IL-12 | | | | | |
| 0 | 34 ± 12 | 44 ± 24 | — | — | 678 ± 125* |
| 10 | — | — | 178 ± 38* | 253 ± 68* | — |
| 100 | — | — | 359 ± 65* | 299 ± 55* | — |
| TNF-α | | | | | |
| 0 | 135 ± 21 | 105 ± 19 | — | — | 1,256 ± 132* |
| 10 | — | — | 245 ± 159* | 259 ± 163* | — |
| 100 | — | — | 377 ± 105* | 489 ± 154* | — |
| IL-10 | | | | | |
| 0 | 14 ± 39 | 54 ± 8 | — | — | 236 ± 22* |
| 10 | — | — | 112 ± 45* | 109 ± 39* | — |
| 100 | — | — | 145 ± 56* | 112 ± 44* | — |
| Nitric Oxide | | | | | |
| 0 | 37 ± 11 | 24 ± 18 | — | — | 921 ± 223* |
| 10 | — | — | 254 ± 44* | 289 ± 55* | — |
| 100 | — | — | 476 ± 52* | 498 ± 74* | — |

Data are expressed as pg/ml, and were obtained from three independent experiments.
*Statistically significant when compared with untreated, L. major-infected control group (P < 0.05).

Compounds of Formula I have Immunostimulatory Effects and Increase Antigen Presenting Cell Activation.

During the development of the present invention, it was found that pyrazine compounds of formula I have an immunostimulatory effect which provides utility outside of, and in addition to the antileishmanial efficacy of the compounds. In particular, compounds of formula I effectively induce immunostimulation of leukocytes. In view of their immunostimulatory effects, compounds of formula I are also useful in the treatment and/or prevention of diseases associated with immunosuppresion of leukocytes, including impaired macrophage function. For example, compounds of formula I are useful in the treatment of, inter alia, HIV, Listeriosis, and various mycobacterium-related diseases (e.g., Mycobacterium tuberculosis complex, Mycobacterium avium complex (MAC), Mycobacterium gordonae clade, Mycobacterium kansasii clade, Mycobacterium nonchromogenicum/terrae clade, Mycolactone-producing mycobacteria, Mycobacterium simiae clade, Mycobacterium chelonae clade, Mycobacterium fortuitum clade, Mycobacterium parafortuitum clade, and Mycobacterium vaccae clade).

Compounds of formula I have an effect on the antigen presenting cell immune function. The elements of the host cell response affected by treatment include phagocytosis, apoptosis, chemotaxis, release of soluble factors and signaling. Treatment affects the activation of macrophages and dendritic cells and the production of cytokines and thereby increases killing of intracellular organisms in L. major-infected mice.

Immune adjuvant discovery. This application goes beyond the evaluation of antileishmanial efficacy of compounds. The mechanism of the efficacy of this compound class in animal models of infection likely involves both parasite and host effects.

Immune responses are required for successful antileishmanial therapy. Leishmania is an intracellular microorganism. The establishment of the appropriate immune response is crucial to parasite elimination and disease prevention. Infection resolves following the activation of antigen presenting cells (macrophages and dendritic cells) to secrete interleukin (IL)-12. This results in the generation and activation of Th(helper)-1 CD4+ T cells and Natural Killer (NK) cells to produce interferon gamma (IFN-γ□□ which stimulates macrophages to increase production of reactive radicals (H₂O₂ and NO). The cytokine Tumor Necrosis Factor alpha (TNF-α□ also causes macrophage activation in an autocrine manner.

The ability of Leishmania to establish infection is dependent upon the immunosuppression of the host, caused by reduction of the production of toxic metabolites, downregulation of signaling and thereby decrease in IL-12 production. This suggests that immunostimulation is required for the successful treatment of leishmaniasis. During development of the present invention, it has been found that compounds of formula I are immunostimulatory and increase antigen presenting cell activation.

Changes in fatty acid metabolism and its relation with immune function. FAs are essential structural and functional cellular components. Alterations in FA metabolism influence cell integrity and/or function. Treatment of macrophages and dendritic cells with compounds of formula I results in the initiation of a proinflammatory response detrimental to diseases associated with immunosuppression of leukocytes, including Leishmania. This effect is believed to be caused by compounds of formula I affecting the host immune response by causing changes in the host cell FA metabolism.

The experimental model: the natural challenge model of L. major. In a “natural” challenge model in C57B1/6 mice that maintains features of natural transmission: intradermal challenge (ear pinna) and low inoculum (≦10⁴) it is possible to examine the mechanism of lesion development, reactivation, and immune response.

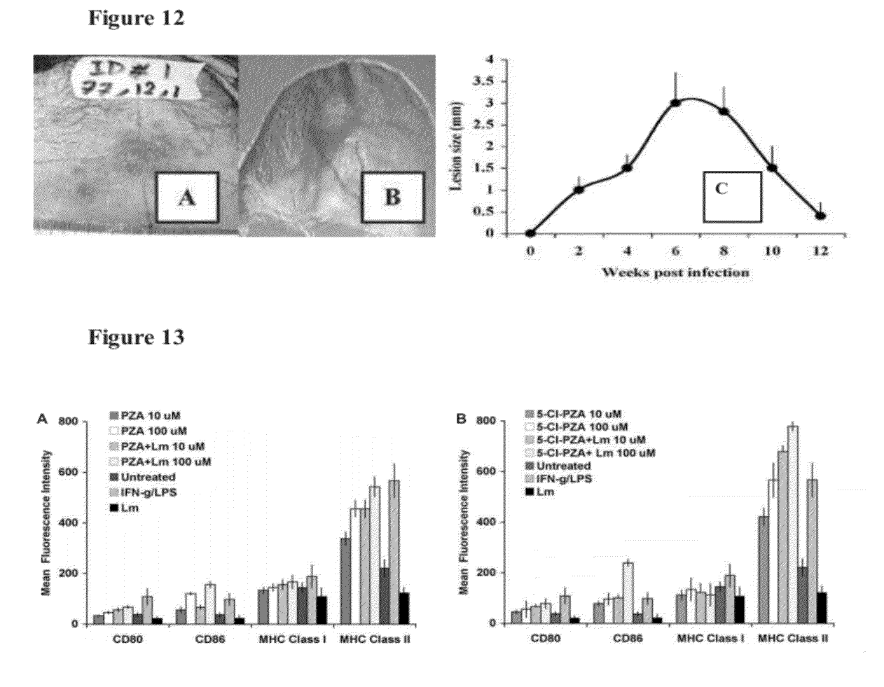

Following inoculation of L. major, C57BL/6 mice develop a skin lesion (FIG. 12B) that is similar to lesions developed in humans (FIG. 12A). These ulcers resolve after 10-12 weeks (FIG. 12C). After inoculation, there is an “immunologically silent” 3-5 week-long interval marked by uncontrolled parasite growth, without formation of a lesion or an inflammatory response. During this silent phase, parasites are preferentially ingested by dermal macrophages. This early “silent phase” is terminated when unchecked parasite growth overwhelms the phagocytic capacity of their macrophages host cells, which lead to dendritic cell uptake. This induces dendritic activation, including upregulation of MHC and co-stimulatory molecules, release of IL-12 and CD4+ T-cell priming, differentiation and polarization, to produce IFN-α. The onset of the protective immune response coincides with the development of a necrotic dermal lesion at the site of inoculation.

Compounds of formula I increase activation of antigen presenting cells to produce IL-12, TNF-α and NO. Because the in vivo leishmanicidal effect of compounds of formula I (PZA and 5-Cl PZA) was greater than what would have been predicted in vitro, the effect of drug treatment on antigen presenting cells was studied during development of the present invention. First, activation markers and cytokine secretion by J774 cells following drug treatment in the presence or absence of L. major infection was studied. As a positive control one group was exposed to a mixture of IFN-γ and LPS. FIG. 13 shows that drug treatment increased expression of the costimulatory molecule CD86 and MHC Class II, in a dose-dependent manner in J774 cells. This effect was enhanced if 5-Cl PZA was employed (FIG. 13B). The same results were obtained in RAW 264.7 cells, which is also a murine macrophage cell line (not shown), indicating that treatment alone increases the ability of the macrophage to present antigen. L. major-infected J774 (or RAW cells) downregulated the expression of costimulatory molecules and Class I and II MHC molecules if compared to uninfected, untreated controls. Treatment of with either PZA or 5-C1 PZA rescued the ability of the cell lines to upregulate activation markers.

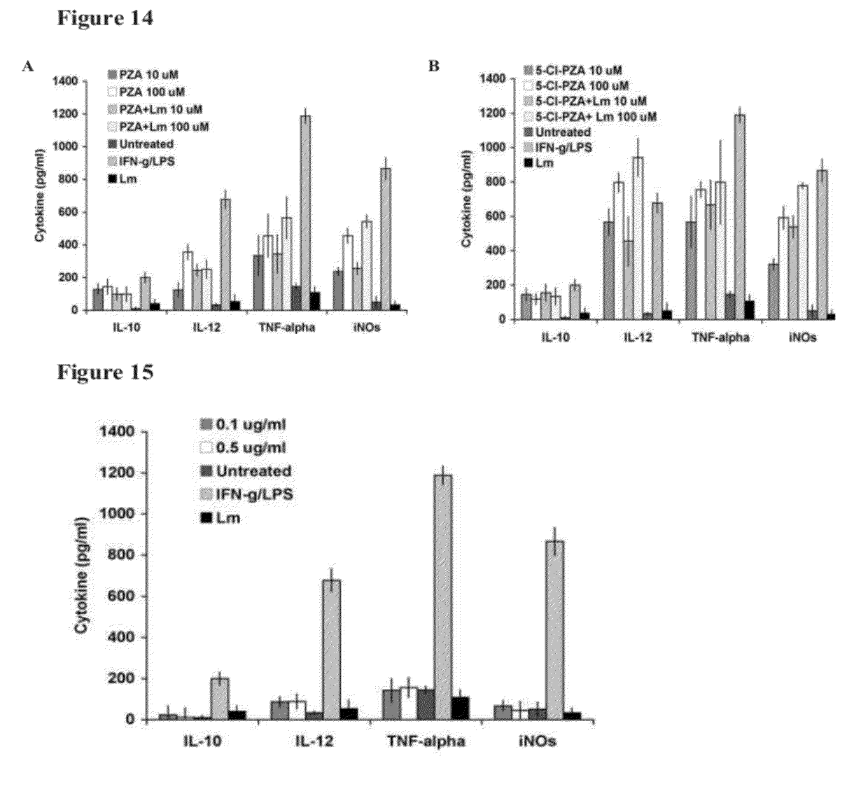

Similarly, production of cytokines, especially proinflammatory IL-12 and TNF-α, as well as NO release, were increased in cells treated with PZA and 5-Cl PZA, irrespective of infection (FIG. 14), indicating that the drugs activate macrophages to initiate an inflammatory response. As before, comparable results were obtained with RAW 246.7 cells. Also as before, macrophages infected with L. major did not release cytokines or NO.

Control experiments. Amphotericin B does not cause macrophage activation. To test the specificity of the immune activation by PZA and 5-C1 PZA, the effect of drug testing on activation and cytokine expression by amphotericin B, one of the current therapeutic agents employed in the treatment against leishmaniasis, was tested. It was shown that treatment of infected cells with 0.1 □g/ml of the antibiotic results in the total elimination of parasites from macrophage cultures. The drug was toxic for mammalian cells at 1 μg/ml. In our system, treatment of J774 or RAW 246.7 cells with the drug did not result in activation at the doses tested, which is highly leishmanicidal (FIG. 15).

Macrophages and bone marrow derived dendritic cells from C57BL/6 mice also produce NO and proinflammatory cytokines upon treatment with PZA and 5-Cl PZA. The murine cell lines employed in studies during development of the instant invention were isolated from the susceptible strain BALB/c mice. Because BALB/c susceptibility to L. major is mediated by its inability to initiate Thl responses, the effect of the drugs on primary cells isolated from the resistant strain C57BL/6 mice was tested.

Bone marrow cells were obtained and were grown in the presence of the cytokines M-CSF or GM-CSF to induce differentiation of macrophages or dendritic cells, respectively. The study of the immune response of dendritic cells was included because of their importance for the initiation of the immune response against L. major. Table III shows that, as before, parasite infection inhibits the initiation of inflammatory responses by macrophages, as evidenced by their inability to produce cytokines or release NO. Again, this effect was rescued by the treatment of macrophages with compounds of formula I, namely, either PZA or 5-Cl PZA. Also as before, the latter compound showed the greatest ability to stimulate macrophage function.

Dendritic cells infected with L. major were able to release IL-12, TNF-α and NO following infection. However, this effect was greatly enhanced (10-fold) if the drugs, especially 5-C1 PZA, were added to the infected cells. Together, these results indicate that compounds of formula I have immunostimulatory properties that contribute to parasite killing and transcend beyond the leishmanicidal effect of the compounds.

TABLE III
IL-12, TNF-α, IL-10 and nitric oxide (NO) production in bone marrow-derived macrophages or dendritic cells
(from C57BL/6 mice) infected or not with L. major and treated with 10 and 100 μM PZA or 5-Cl PZA.
Uninfected cells were treated with 100 ng/ml LPS and 10U IFN-γ as a positive control. Cytokine levels
in unstimulated cells are also shown.

| | | | | | | |
|---|---|---|---|---|---|---|
| Unstimulated | Lm | LPS + IFN-γ | PZA | PZA + Lm | 5-Cl PZA | 5-Cl PZA + Lm |

Macrophage

| | | | | | | | |
|---|---|---|---|---|---|---|---|
| IL-12 | | | | | | | |
| No drug | 56 ± 11 | 34 ± 6 | 3678 ± 456 | — | — | — | — |
| 100 μM | — | — | — | 867 ± 546 | 921 ± 445 | 1767 ± 526 | 1821 ± 645 |
| IL-10 | | | | | | | |
| No drug | 15 ± 13 | 30 ± 22 | 321 ± 156 | — | — | — | — |
| 100 μM | — | — | — | 155 ± 21 | 199 ± 120 | 415 ± 210 | 499 ± 312 |
| TNF-α | | | | | | | |
| No drug | 35 ± 22 | 104 ± 89 | 2678 ± 625 | — | — | — | — |
| 100 μM | — | — | — | 758 ± 246 | 921 ± 345 | 1598 ± 542 | 1928 ± 444 |
| NO | | | | | | | |
| No drug | 30 ± 3 | 12 ± 21 | 1240 ± 516 | — | — | — | — |
| 100 μM | — | — | — | 543 ± 221 | 699 ± 112 | 1145 ± 721 | 1599 ± 812 |

Dendritic cell

| | | | | | | | |
|---|---|---|---|---|---|---|---|
| IL-12 | | | | | | | |
| No drug | 46 ± 31 | 114 ± 26 | 3365 ± 789 | — | — | — | — |
| 100 μM | — | — | — | 956 ± 145 | 1035 ± 785 | 1962 ± 765 | 2121 ± 945 |
| IL-10 | | | | | | | |
| No drug | 35 ± 33 | 160 ± 52 | 621 ± 102 | — | — | — | — |
| 100 μM | — | — | — | 185 ± 63 | 203 ± 60 | 265 ± 213 | 342 ± 398 |
| TNF-α | | | | | | | |
| No drug | 102 ± 45 | 637 ± 67 | 3456 ± 768 | — | — | — | — |
| 100 μM | — | — | — | 1654 ± 546 | 1856 ± 125 | 17684 ± 678 | 2986 ± 566 |
| NO | | | | | | | |
| No drug | 156 ± 39 | 243 ± 71 | 806 ± 506 | — | — | — | — |
| 100 μM | — | — | — | 545 ± 221 | 699 ± 612 | 745 ± 211 | 876 ± 172 |

### The Stimulatory Effect of PZA is Independent of TLR Engagement

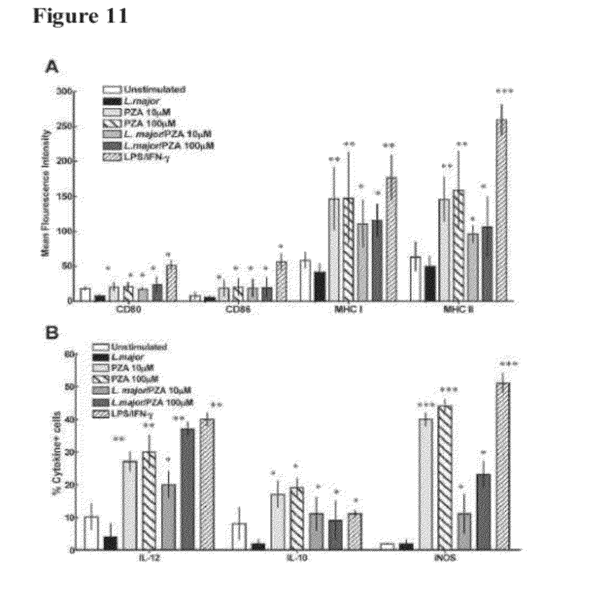

To confirm that the immunostimulatory effect of PZA is directly caused by the drug and not due to contaminants (i.e. endotoxin) or other ligands that could cause cell activation via TLRs, we studied the effect of drug treatment on bone marrow derived dendritic cells isolated from TLR-2/-4 double knock out mice. In this experiment, we treated the dendritic cells as described above, in the presence or absence of L. major infection. Again as a positive control, cells were primed with IFN-γ and LPS. FIG. 11A shows that drug treatment increased expression of activation markers and MHC Class molecules in a dose-dependent manner in dendritic cells lacking TLR-2 and -4, suggesting that PZA treatment, and not other contaminant, was responsible for cell activation. As before, infected cells treated with PZA showed an increase in upregulation of activation markers compared to cells treated with L. major alone. In the same way, the expression of IL-12, IL-10 and iNOS was increased in treated cells, irrespective of infection (FIG. 11B). These data further confirmed that PZA activates dendritic cells to initiate an inflammatory response, and that this is a direct effect caused by the compound.

### Discussion

The emergence of the leishmaniases, and the lack of affordable therapy, have necessitated the development of novel antileishmanial therapies. We have shown that the clinical drug PZA has both in vivo and in vitro activity against L. major. PZA is a drug that has been employed extensively, first used in the treatment of pulmonary tuberculosis in humans in 1949. The use of this licensed, well-known drug for indications other than the treatment of tuberculosis could eliminate hurdles associated with the development of new anti-leishmanial antigens and provide therapeutic alternatives for a disease in which chemotherapy is suboptimal. Moreover, PZA is an orally administered drug, therefore obviating the need for parenteral injections.

Our data show that PZA is very efficient in controlling the growth of L. major in vitro. The MIC₅₀ is estimated to be 10 μM for promastigotes and 100.1 μM for amastigotes. These MIC₅₀ values are comparable to what was obtained by Klemens et al. in a murine model of tuberculosis. Although intracellular amastigotes appeared to be less sensitive than promastigotes to the effect of the PZA at 48 hours post infection (10-fold increase in MIC₅₀), an extended kinetic analysis revealed that PZA, employed at 100 μM, eliminated 90% of L. major in cultured cells after 120 hours of culture. This concentration is equivalent to what was found by Zhu et al. in pharmacokinetic studies of PZA-treated children (serum concentration was 41 μg/ml), indicating that the standard antituberculous treatment regimen will be appropriate for the control of L. major infections. Our in vivo data demonstrate that PZA treatment significantly diminished lesion development in mice infected with L. major at all concentrations tested (900, 450 and 150 mg/ml). PZA treatment also significantly reduced parasite burden in the infection site without compromising the overall health of the infected mice.

Our results demonstrate that long-term treatment of leishmanial cultures with PZA resulted in near complete elimination of parasites from macrophages. If fatty acid synthesis is non-essential in Leishmania, or these organisms are highly resistant to lipid depletion, it is possible that parasite killing is not exclusively mediated by the direct effect of the drug on parasite survival and replication. Thus, the anti-leishmanial effect of PZA may be caused (or enhanced) by chemotherapeutic interaction with the macrophage. Our data showing that J774 cells, as well as primary cells from C57BL/6 mice, upregulate activation markers and release cytokines following treatment with PZA, indicates that the drug enhances immune response to L. major infection. This immunoenhancing effect could not be replicated in cells treated with amphotericin B, despite publications reporting the immunostimulatory effect of the drug, or in cells deficient in TRL-2 and -4 receptors, confirming that immunostimulation is a PZA-specific event. This phenomenon would be especially desirable in situations where patients are immunocompromised. Because Leishmania/HIV co-infections have been extensively documented, the development of drugs that boost the immune system of the host may be extremely useful.

### Materials and Methods

Mice. C57BL/6 mice (5-6 weeks of age) were purchased from Taconic (Germantown, N.Y.). All mice were maintained in the Baker Institute Animal Care Facility under pathogen-free conditions.

Parasite and cell culture. L. major clone V1 (MHOM/IL/80/Friedlin) promastigotes were grown at 26° C. in medium 199 supplemented with 20% heat-inactivated fetal calf serum (FCS, Gemini, Sacramento), 100 U/ml penicillin, 100 μg/ml streptomycin, 2 mM L-glutamine, 40 mM Hepes, 0.1 mM adenine (in 50 mM Hepes) and 5 mg/ml hemin (in 50% triethanolamine). The macrophage murine cell line J774 (Cat. No. TIB-67TM) was cultured in DMEM (Sigma-Aldrich, St. Louis, Mo.) with 10% FCS, 100 U/ml penicillin, 100 μg/ml streptomycin and 2 mM glutamine (Sigma-Aldrich) at 37° C. under 5% CO₂ atmosphere. Culture medium was changed twice per week. Subcultures were performed when monolayers covered 90% of the bottom of culture flasks. For experiments involving macrophages and dendritic cells, bone marrow was obtained from C57BL/6 mouse femurs and grown for 6-8 days in RPMI 1640 supplemented as above in the presence of 10% L929 conditioned medium (to generate macrophages) or 20 ng/ml GM-CSF (to generate dendritic cells).

### Promastigote and Amastigote Drug Treatment.

Mid-log phase (day 3 of culture) L. major promastigotes were employed. Parasite concentration was adjusted to 10⁶ promastigotes/ml and seeded into 96-well plates in a volume of 100 μl (final concentration, 10⁵ promastigotes/well). PZA was tested in triplicate in a concentration gradient from 1,000-0.5 μg/ml and added to the wells containing the parasite in a volume of 100 μl. A negative control was included with three wells containing only parasites and medium. The positive control consisted of amphotericin B (1 μg/ml). This concentration was previously employed by us because this concentration is equivalent to drug concentrations achieved in human plasma. After 48 h of incubation at 26° C., 10 μL of each well was diluted in 90 μL of the vital colorant (trypan blue in PBS) and the parasites were quantified in a Neubauer chamber. Data was normalized as percentage of survival compared to untreated controls. The Lethal Dose 50 (LD50) was extrapolated from the graph as the concentration of the products that inhibited the parasitic growth at 50% of the values of the negative control.

Amastigotes were generated by infecting J774 murine macrophages. Infections were carried after seeding cells in 8-well Labtek chambers (Thermo Physic Scientific, Rochester N.Y.) at a concentration of 5×10⁴ cells/well. To avoid multiplication, cells were incubated with mitomycin C at a concentration of 0.8 μg/ml for 16 h. Infective-stage promastigotes (metacyclics) of L. major were isolated from stationary cultures (4-5 day-old) by Ficoll enrichment, added to macrophage cultures (5 promastigotes: 1 cell) and kept overnight at 37° C. in the presence of 5% CO₂ and DMEM with 10% FCS. Sixteen hours later, non-internalized promastigotes were then washed and replaced by culture medium containing the drug. Forty-eight hours later, wells were detached from the slides, stained with Diff-Quick (Dade Behring, Newark, Del.) and counted under light microscope. Parasite burden was determined by observation under light microscopy (1000×) as the number of amastigotes per one hundred J774 cells. Percentage of survival of amastigotes and MIC₅₀ were calculated as above.

J774 cell viability following incubation with PZA was also determined. Cells were seeded onto 8-well Labtek chambers as above and incubated with different concentrations of PZA (up to 1 mg/ml) for 48 h, stained with Diff-Quick and counted under light microscope. The percentage of viable cells was determined after quantifying the number of cells present per field (in 25 fields, ca. 500 cells). Percentage of survival and MIC₅₀ were calculated as above.

### In Vivo Infection Studies

Mice (n=6) were anesthetized with isoflurane (Abbott Laboratories, Chicago, Ill.) and vaccinated intradermally in both ears with 5×10⁵ L. major promastigotes in a volume of 10 μl using a 27½ G needle. PZA was diluted in water and administered by oral gavage in a 0.2 ml volume. A control group was treated with water containing DMSO (3.8%). The timetable for the experiment was as follows: day 0, infection; days 1-5, 8-12, 15-19 and 22-26, drug administration, and day 70, sacrifice. Lesion size was monitored 1-2 times per week by measuring the lesion diameter with a vernier caliper. Mice were sacrificed by CO₂ inhalation.

### Parasite Quantitation

Parasite loads in the ears were determined as previously described. Briefly, the ventral and dorsal sheets of the infected ears were separated and deposited in RPMI containing 100 U/ml penicillin/100 μg/ml streptomycin and Liberase CI enzyme blend (Boehringer Mannheim, 0.5 mg/ml). Ears were incubated for 60 min at 37° C. The sheets were dissociated using a handheld tissue homogenizer. The homogenates were filtered using a 70 μm an cell strainer (BD Falcon, San Jose, Calif.) and serially diluted in a 96-well flat bottom microtiter plate containing biphasic medium prepared using 50 μl NNN medium containing 20% of defibrinated rabbit blood overlaid with 100 μl medium 199. The number of viable parasites in each ear was estimated from the highest dilution at which promastigotes could be grown out after 7 days of incubation at 26° C.

### Analysis of J774 Activation

J774 cells or bone marrow-derived cells were seeded onto 24-well plates at a concentration of 5×10⁵ cells/ml. Twenty-four hours later, PZA was added to the wells at different concentrations in the absence or presence of L. major (1:5 parasite ratio). In some experiments, J774 cells were treated with amphotericin B (0.1, 0.5, or 1 μg/ml). Two control groups were also included in this experiment: a positive control of activation consisting in a group of uninfected cells treated with 100 ng/ml LPS and 10 IU IFN-γ as a positive control of activation, and a negative control of activation, consisting in uninfected, untreated cells. Cell cultures were maintained overnight, and then cultured for an additional 6 h with brefeldin A (10 μg/ml), harvested by scraping and fixed in 2% paraformaldehyde. Prior to staining, cells were incubated with an anti-Fc III/II receptor and 10% normal mouse serum (NMS) in PBS containing 0.1% BSA, 0.01% NaN₃. Cells were permeabilized with saponin and stained for the surface markers CD80 (clone 16-10A1), CD86 (clone GL1), MHCI (clone 28-14-8), MHCII (clone M5/114.15.2) and for the cytokines IL-12p40/p70 (clone C17.8) and IL-10 (clone JESS-16E3). Incubations were carried out for 30 min on ice. All antibodies were purchased from BD Biosciences or eBioscience. For each sample, at least 50,000 cells were analyzed. The data were collected and analyzed using CELLQuest software and a FACScalibur flow cytometer (Becton Dickinson, San Jose, Calif.).

### Statistical Analysis

Statistical analysis of the in vivo data used a one-way ANOVA and compared with the control by Dunnet's test using GraphPad Prism (San Diego, Calif.). Results were considered significant when P<0.05. MIC₅₀ values were interpolated from curves generated by non-lineal regression analysis using GraphPad Prism.

1. A method of treating leishmaniases or a disease or disorder caused by Trypanosoma cruzi or Trypanosoma brucei, comprising administering to a patient in need thereof a pharmaceutically effective amount of a pyrazine compound of formula I: 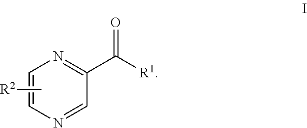
or a salt thereof, wherein
R¹ is chosen from NR⁴R⁵ and OR³;
R² is chosen from H and halogen;
R³ is chosen from H and C₁ to C₂₀ alkyl; and
R⁴ and R⁵ are individually chosen from H, NH₂, C₁ to C₂₀ alkyl, oxaalkyl, and heterocyclylalkyl, or taken together R⁴ and R⁵, together with the nitrogen to which they are attached, form a heterocyclic ring.

2. A method according to claim 1 for treating leishmaniases.

3. A method according to claim 2 comprising use of a combination of a compound of formula I or a salt thereof and one or more compounds or salts thereof selected from the group consisting of:
(a) meglumine antimoniate;
(b) sodium stibogluconate;
(c) amphotericin B;
(d) paromomycin;
(e) pentamidine;
(f) miltefosine; and
(g) ketoconazole.

4. A method according to claim 1 for treating or preventing a disease or disorder caused by Trypanosoma cruzi.

5. A method according to claim 4, comprising use of a combination of a compound of formula I or a salt thereof and one or more compounds or salts thereof selected from the group consisting of:
(a) nifurtimox; and
(b) benznidazole.

6. A method according to claim 1 for treating or preventing a disease or disorder caused by Trypanosoma brucei.

7. A method according to claim 6, comprising use of a combination of a compound of formula I or a salt thereof and one or more compounds or salts thereof selected from the group consisting of:
(a) nifurtimox;
(b) pentamidine;
(c) eflornithine; and
(d) melarsoprol.

8. A method according to claim 1 wherein the pyrazine compound of formula I is pyrazinamide or a salt thereof

9. A method according to claim 1 wherein R¹ is NH₂.

10. A method according to claim 9 wherein R² is halogen.

11. A method according to claim 10 wherein the pyrazine compound is 5-chloro pyrazinamide.

12. A method according to claim 1 wherein at least one of R⁴ and R⁵ is morpholinomethyl.

13. A method according to claim 1 wherein the pyrazine compound is a compound of:
(a) Formula IA: 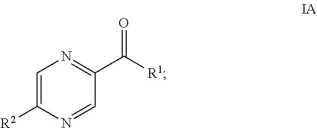
(b) Formula IB: 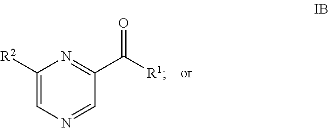
(c) Formula IC:

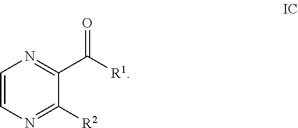

14. A method of inducing immunostimulation of leukocytes in a patient suffering from a disease or condition associated with immunosuppression of leukocytes comprising administering to the patient a pharmaceutically effective amount of a pyrazine compound of formula I: 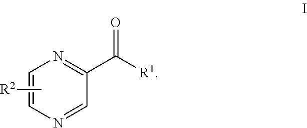
or a salt thereof, wherein
R¹ is chosen from NR⁴R⁵ and OR³;
R² is chosen from H and halogen;
R³ is chosen from H and C₁ to C₂₀ alkyl; and
R⁴ and R⁵ are individually chosen from H, NH₂, C₁ to C₂₀ alkyl, oxaalkyl, and heterocyclylalkyl, or taken together R⁴ and R⁵, together with the nitrogen to which they are attached, form a heterocyclic ring.

15. A method according to claim 14 wherein the pyrazine compound of formula I is pyrazinamide or a salt thereof.

16. A method according to claim 14 wherein R¹ is NH₂.

17. A method according to claim 16 wherein R² is halogen.

18. A method according to claim 17 wherein the pyrazine compound is 5-chloro pyrazinamide.

19. A method according to claim 14 wherein at least one of R⁴ and R⁵ is morpholinomethyl.

20. A method according to claim 14 wherein the pyrazine compound is a compound of:
(a) Formula IA: 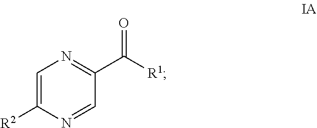
(b) Formula IB: 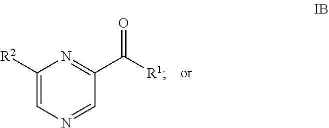
(c) Formula IC: [chemical structure]
